(19) United States
(12) Patent Application Publication
(10) Pub. No.: US 2021/0096096 A1
(43) Pub. Date: Apr. 1, 2021

(54) SENSOR ELECTRODE, SENSOR, AND METHOD OF PRODUCTION

(71) Applicant: KING ABDULLAH UNIVERSITY OF SCIENCE AND TECHNOLOGY, Thuwal (SA)

(72) Inventors: Yongjiu LEI, Thuwal (SA); Husam Niman ALSHAREEF, Garland, TX (US)

(21) Appl. No.: 16/970,738

(22) PCT Filed: Feb. 12, 2019
(86) PCT No.: PCT/IB2019/051132

§ 371 Date: Aug. 18, 2020

Related U.S. Application Data
(60) Provisional application No. 62/665,600, filed on May 2, 2018.
(60) Provisional application No. 62/639,144, filed on Mar. 6, 2018.

(51) Int. Cl. G01N 27/327; C12Q 1/00; G01N 27/30

(52) U.S. Cl. CPC G01N 27/3277; G01N 27/3278; G01N 27/308; C12Q 1/005

(57) ABSTRACT

An electrode includes a substrate and a composite arranged on the substrate. The composite includes MXene and Prussian blue.

### CROSS-REFERENCE TO RELATED APPLICATIONS

This application claims priority to U.S. Provisional Patent Application No. 62/639,144, filed on Mar. 6, 2018, entitled “HYDROGEN PEROXIDE SENSOR USING TERNARY ELECTRODE COMPRISING MXENE-PRUSSIAN-CNT COMPOSITES,” and U.S. Provisional Patent Application No. 62/665,600, filed on May 2, 2018, entitled “SENSOR ELECTRODE, SENSOR, AND METHOD OF PRODUCTION,” the disclosures of which are incorporated herein by reference in their entirety.

### BACKGROUND

### Technical Field

Embodiments of the disclosed subject matter generally relate to a sensor electrode including a composite of MXene and Prussian Blue, a sensor including such a sensor electrode, and a method of production.

### Discussion of the Background

Hydrogen peroxide (H₂O₂) is a molecule of great importance in pharmaceutical, clinical, environmental and food manufacturing applications. Hydrogen peroxide is also a side product generated from a number of biochemical reactions catalyzed by enzymes, such as glucose oxidase, lactate oxidase, alcohol oxidase, urate oxidase, cholesterol oxidase. The importance of hydrogen peroxide in biological field and its practical applications requires development of hydrogen peroxide sensors exhibiting high sensitivity and good stability in their measurement environment. Common hydrogen peroxide detection techniques, including fluorimetry, chemiluminescence, fluorescence, and spectrophotometry, are complex, costly, and not portable.

Because hydrogen peroxide is an electroactive molecule, investigations have been performed to build an electrochemical hydrogen peroxide sensor, which is simple, rapid, sensitive, and cost effective. Prussian blue (also referred to as potassium ferric hexacyanoferrate), is one of the most commonly used electrochemical mediator to detect hydrogen peroxide because Prussian blue can detect hydrogen peroxide at an applied potential around 0 V vs. Ag/AgCl, which reduces or avoids electrochemical interference. Prussian blue, however, exhibits low stability under basic pH and low conductivity, both of which limit its performance in a practical application as a hydrogen peroxide sensor.

Accordingly, research has been performed to identify supports for Prussian blue that can improve its stability and conductivity. Most research have focused on carbon nanotubes (CNTs) and graphene because these materials both exhibit unique stability and good conductivity. Forming sensors with working electrodes comprising composites of Prussian blue and carbon nanotubes or Prussian blue and graphene involves a multistep process to prepare the carbon nanotubes or graphene. Furthermore, Prussian blue/carbon nanotube and Prussian blue/graphene composites exhibit limited sensitivity to hydrogen peroxide, which limits their ability to be used in many practical applications.

Thus, it would be desirable to provide working electrodes comprising Prussian blue that exhibit high sensitivity to hydrogen peroxide. It would also be desirable to provide sensors having working electrodes comprising Prussian blue that exhibit high sensitivity to hydrogen peroxide.

### SUMMARY

According to an embodiment, there is an electrode, which includes a substrate and a composite arranged on the substrate. The composite includes MXene and Prussian blue.

According to another embodiment, there is a method of forming an electrode. A composite of MXene and Prussian blue is formed and arranged on a substrate.

According to a further embodiment, there is a sensor, which includes a voltage source, a reference electrode coupled to the voltage source, a counter electrode coupled to the voltage source, a working electrode comprising a substrate and a composite comprising MXene and Prussian blue on the substrate, and a current meter coupled to the working electrode.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1A is a schematic diagram of an electrode according to an embodiment;

FIG. 1B is a transmission electron microscope (TEM) image of a composite of MXene and Prussian blue according to an embodiment;

FIG. 10 is a scanning electron microscope image of a composite of MXene and Prussian blue combined with a binder according to an embodiment;

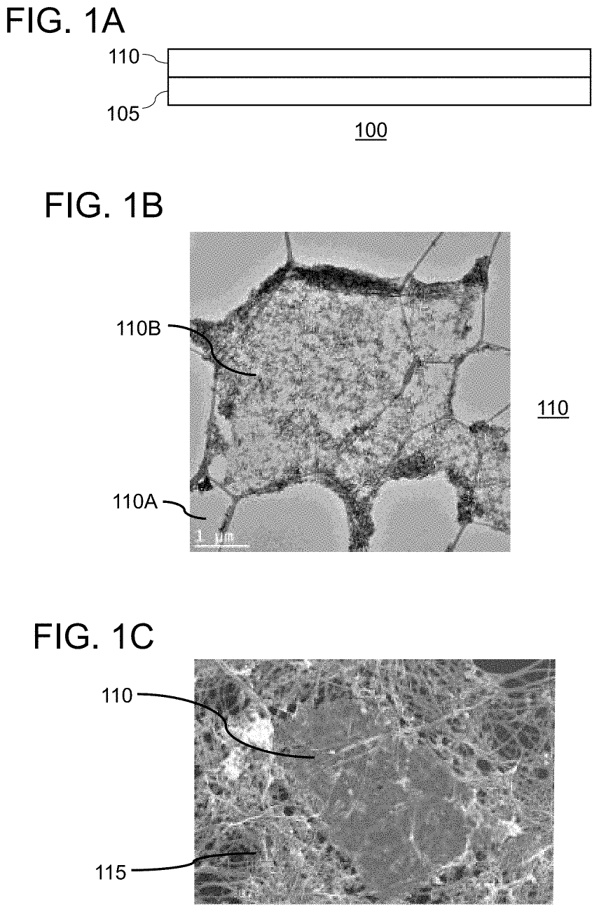

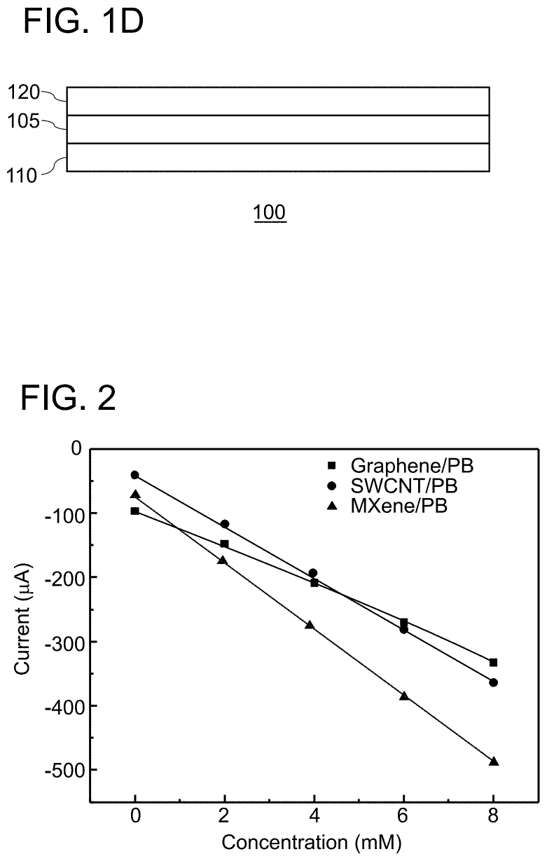

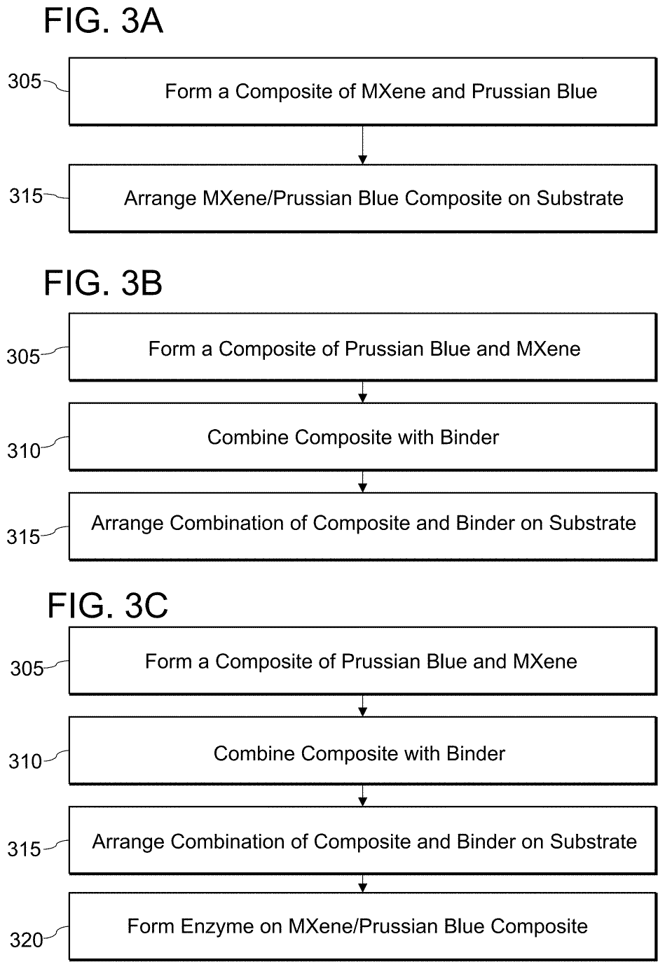

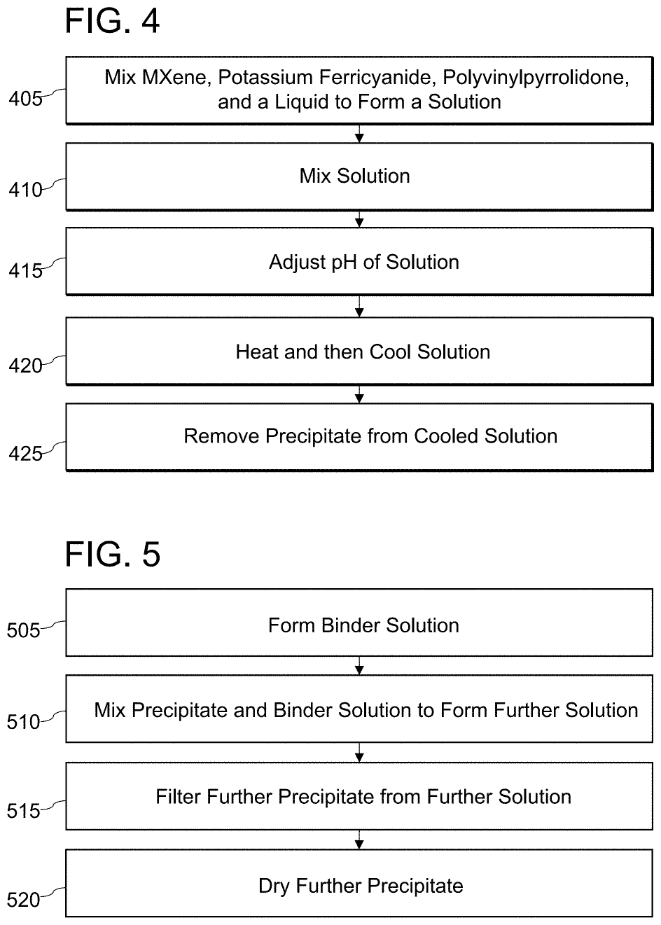

FIG. 1D is a schematic diagram of an electrode according to an embodiment;

FIG. 2 is a current versus concentration graph of the electroreduction of hydrogen peroxide according to an embodiment;

FIGS. 3A-3C are flowcharts of methods for forming an electrode according to embodiments;

FIG. 4 is a flowchart of a method of making a composite of MXene and Prussian blue according to an embodiment;

FIG. 5 is a flowchart of a method of combining a composite of MXene and Prussian blue with a binder according to an embodiment;

FIG. 6 is a schematic diagram of a hydrogen peroxide sensor according to an embodiment;

FIG. 7A is a schematic diagram of a glucose sensor without a protective cover according to an embodiment;

FIG. 7B is a schematic diagram of a glucose sensor having a protective cover according to an embodiment; and

FIG. 7C is a schematic diagram of a glucose measurement system according to an embodiment.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of electrochemical sensors.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

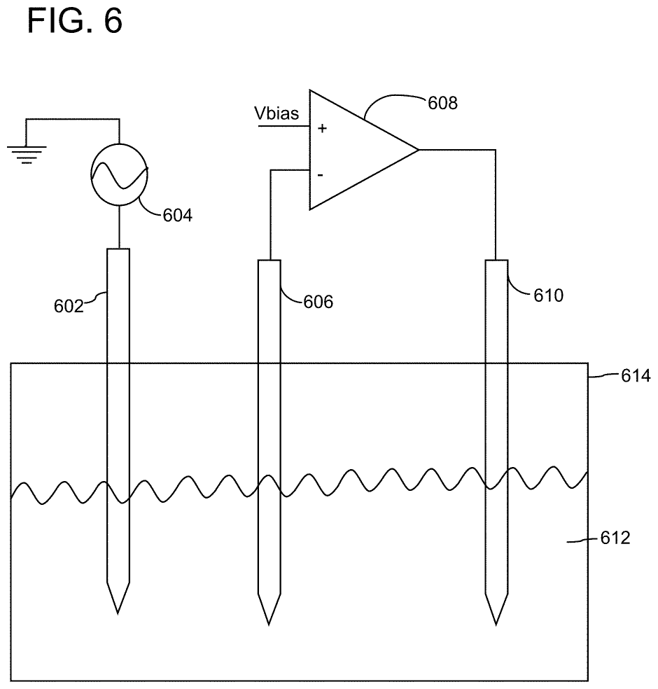

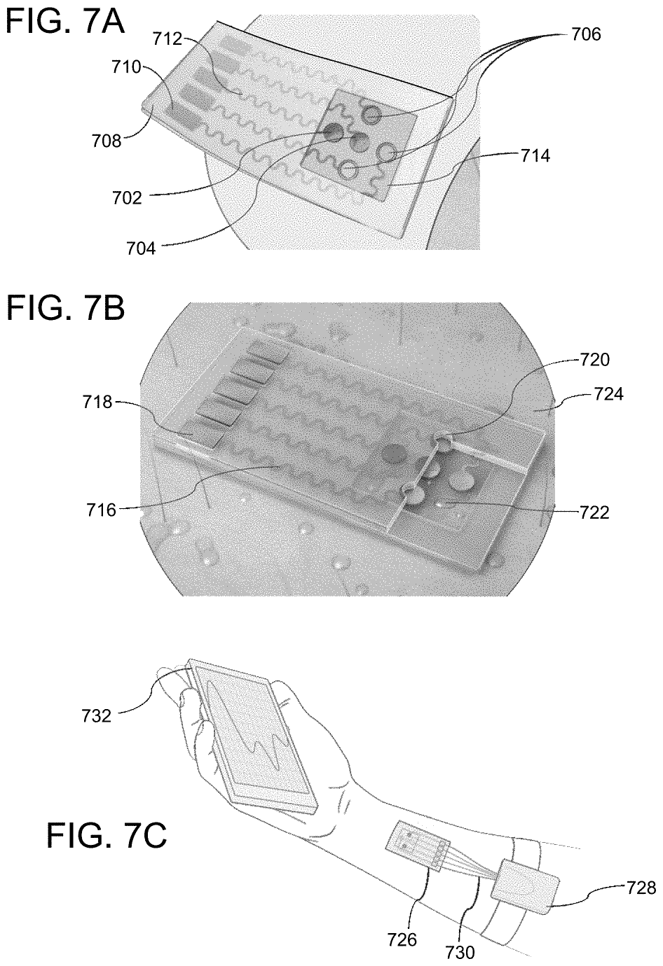

Referring now to FIGS. 1A-1C, an electrode 100 includes a substrate 105 and a composite 110 arranged on the substrate 105. The composite 110 includes MXene 110A and Prussian blue 110B. In an embodiment, the substrate can be, for example, carbon fiber. A binder can be provided in order to improve the adhesion of the MXene/Prussian blue composite 110 to substrate 105. For example, FIG. 10 illustrates a carbon nanotube binder 115 combined with the MXene/Prussian blue composite 110. Using a carbon nanotube binder 115 not only improves the adhesion of the MXene/Prussian blue composite 110 to the substrate 105, the carbon nanotube binder can also improve the conductivity of the MXene/Prussian blue composite 110.

It will be recognized that MXenes are a class of two-dimensional inorganic compounds that include layers that are a few atoms thick of transition metal carbides, nitrides, and carbonitrides. In one embodiment, the MXene used in the MXene/Prussian blue composite 110 is Ti₃C₂Tₓ, which exhibits very good conductivity, i.e., the conductivity of Ti₃C₂Tₓ ranges from 1,000 Scm⁻¹ to 6,500 Scm⁻¹. Other MXenes can be employed instead of Ti₃C₂Tₓ. Because not all MXenes exhibit the same conductivity as Ti₃C₂Tₓ, the carbon nanotube binder can improve the conductivity of an MXene/Prussian blue composite 110 in which the MXene is not Ti₃C₂Tₓ.

The electrode 100 having an MXene/Prussian blue composite 110 exhibits very good sensitivity to hydrogen peroxide, and thus can be used in a variety of applications. Specifically, the electrode 100 has a detection limit of approximately 200 nano Molar and limited detection from 50 nano Molar at a signal-to-noise ratio of 3.

Further applications can be achieved by providing an enzyme on the MXene/Prussian blue composite 110, an example of which is illustrated in FIG. 1D in which an enzyme 120 is arranged on the MXene/Prussian blue composite 110. The enzyme 120 forms hydrogen peroxide by catalysis with an enzyme, such as glucose oxidase (used for detecting glucose concentrations), lactate oxidase (used for detecting lactose concentrations), alcohol oxidase (used for detecting alcohol concentrations), urate oxidase (used for detecting uric acid concentrations), choline oxidase (used for detecting choline), and cholesterol oxidase (used for detecting cholesterol concentrations). These enzymes are merely examples of enzymes that can be used with the disclosed electrode 100 and other enzymes can be employed. Thus, the electrode without an enzyme can directly detect the presence of hydrogen peroxide in a solution and the addition of an enzyme allows detection of hydrogen peroxide as a byproduct of a reaction of the enzyme. Accordingly, the reaction of glucose with glucose oxidase, of lactate with lactate oxidase, alcohol with alcohol oxidase, uric acid with urate oxidase, choline with choline oxidase, and cholesterol with cholesterol oxidase all produce hydrogen peroxide, the level of which indicates the concentration of glucose, lactate, alcohol, uric acid, choline, or cholesterol in a solution, such as, for example, in sweat.

The electrode having an MXene/Prussian blue composite 110 with a carbon nanotube binder 115 exhibits significantly better sensitivity compared to Prussian blue with a carbon nanotube binder and graphene/Prussian blue composites with a carbon nanotube binder, which is reflected in the graph of FIG. 2. The graph of FIG. 2 is a calibration plot derived from a cyclic voltammetry at −0.1 V versus Ag/AgCl of a graphene/Prussian blue composite with a carbon nanotube binder (the plot with the squares in the figure), single wall carbon nanotubes (SWCNT)/Prussian blue composite (the plot with the circles in the figure), and MXene/Prussian blue composite with a carbon nanotube binder (the plot with the triangles in the figure). As illustrated, the graphene/Prussian blue composite with a carbon nanotube binder produces currents ranging from approximately −100 pA at a zero concentration of hydrogen peroxide to approximately −350 pA at a concentration of 8 mM of hydrogen peroxide; the SWCNT/Prussian blue composite produces currents ranging from approximately −25 pA at a zero concentration of hydrogen peroxide to approximately −375 pA at a concentration of 8 mM of hydrogen peroxide; and the MXene/Prussian blue composite with a carbon nanotube binder produces currents ranging from approximately −75 pA at a zero concentration of hydrogen peroxide to approximately −500 pA at a concentration of 8 mM of hydrogen peroxide. Thus, over this concentration range, the graphene/Prussian blue composite with a carbon nanotube binder has a change in current response of approximately 250 pA, the SWCNT/Prussian blue composite has a change in current response of approximately 350 pA, and MXene/Prussian blue composite with a carbon nanotube binder has change in current response of approximately 425 pA. The significantly larger change in current response over the range of hydrogen peroxide concentrations of the MXene/Prussian blue composite with a carbon nanotube binder compared to the graphene/Prussian blue composite with a carbon nanotube binder and SWCNT/Prussian blue composite allows for a more granular measurement of hydrogen peroxide concentrations because there is a greater range of current values corresponding to the range of concentrations.

Methods for forming an electrode having an MXene/Prussian blue composite will now be described in connection with FIGS. 3A-3C. Initially, a composite of an MXene and Prussian blue is formed (step 305). The MXene/Prussian blue composite is then arranged on a substrate (step 315).

As discussed above, adhesion between the MXene/Prussian blue composite and the substrate can be improved by using a binder. In this case, after the MXene/Prussian blue composite is formed (step 305), the MXene/Prussian blue composite is combined with a binder (step 310). The combination of the MXene/Prussian blue composite and binder is then arranged on a substrate (step 315).

As also discussed above, use of the electrode comprising the MXene/Prussian blue composite can be expanded by including an enzyme on the MXene/Prussian blue composite. In this case, after the combination of the MXene/Prussian blue composite and binder are arranged on a substrate (step 315), the enzyme can be formed on the MXene/Prussian blue composite (step 320). Although the method of FIG. 3C describes the enzyme being formed in the last step, the enzyme can also be formed after combining the composite with the binder (step 310) and the arrangement of the composite and binder on the substrate (step 315). This alternative method of forming the enzyme, however, results in a lower device performance compared to forming the enzyme as the final step.

The MXene/Prussian blue composite can be formed using any technique, one of which will be described in connection with FIG. 4. Initially, MXene, potassium ferricyanide, polyvinylpyrrolidone, and a liquid are mixed to form a solution (step 405). This can involve, for example, dissolving 5 mg of MXene nanoflakes (having a size of approximately 1-4 micron), 30 mg of potassium ferricyanide, and 100 mg of polyvinylpyrrolidone in 10 ml of deionized water, which produces a dark brown solution. The solution is then mixed (step 410), which can involve, for example, bubbling the solution with nitrogen or argon for more than 30 minutes.

The pH of the solution is then adjusted (step 415), which can be achieved, for example, by adding a hydrogen chloride acid aqueous solution (6 molL⁻¹) to achieve a pH of 2.0. The pH adjusted solution can then be heated and subsequently cooled (step 420). The heating and cooling can involve, for example, sealing the pH adjusted solution in an autoclave, heating the autoclave to 70° C. and maintaining the temperature for two hours and then allowing the autoclave to cool down to room temperature. The resulting suspension can appear dark blue. Further, the pH adjustment can be performed while the solution is in the autoclave but before the autoclave is sealed.

Finally, the precipitate is removed from the cooled solution (step 425). This can be achieved, for example, by centrifuging the solution. Furthermore, the precipitate can be washed and then added to liquid to form a solution. For example, the precipitate can be washed three times with deionized water and then dissolved in deionized water.

Combining the MXene/Prussian composite with the binder (e.g., carbon nanotubes) can be achieved using any technique, one of which will be described in connection with FIG. 5. Initially, a binder solution is formed (step 505). This can involve, for example, preparing a carbon nanotube solution having a concentration of 0.1 mg/mL by dissolving 50 mg of carbon nanotubes and 500 mg of sodium dodecyl sulfate in 500 mL of deionized water, and then sonicating the suspension, for example for 20 hours and then centrifuging the solution. The MXene/Prussian composite precipitate is then mixed with the binder solution to form a further solution (step 510). This can be achieved, for example, by dissolving 120 microliters of the MXene/Prussian composite precipitate having a concentration of 5 mg/mL with 2 mL of the carbon nanotube binder solution having a concentration of 0.1 mg/mL in 200 mL of deionized water to form a further precipitate in the further solution. The enzyme can be added to the 200 mL along with the MXene/Prussian composite and carbon nanotube binder, or the enzyme can be formed at the end of this method consistent with the method of FIG. 3C. The further precipitate is then filtered from the further solution, which forms a thin film (step 515). The filtering can be, for example, vacuum filtration. The filtered further precipitate in the form of a thin film, which forms the working electrode, can then be dried prior to use (step 520). The thickness of the thin film working electrode can be, for example, between 0.1 μm and 1.0 μm. It has been recognized that the thickness of the thin film significantly affects performance of the electrode and that the aforementioned thickness provides optimal mechanical and electrochemical performance. After drying, the working electrode can then be shaped, for example by cutting, into any desired form. The thin film forming the working electrode comprises a plurality of layers of the composite of MXene and Prussian blue, which layers are held together by the binder.

FIG. 6 is a schematic diagram of a sensor according to an embodiment. The sensor includes a working electrode 602 coupled to a current meter 604. The sensor also includes a reference electrode 606 coupled to a negative input to an operational amplifier 608 and a counter electrode 610 coupled to an output of the operational amplifier 608. The reference electrode 606 can be comprised of, for example, silver/silver chloride and the counter electrode 610 can be, for example, a platinum wire.

A voltage source V_{bias} is coupled to the positive input of the operational amplifier 608. The voltage source V_{bias} produces a voltage that is close to 0 V, for example, −0.1 V. The working electrode 602, reference electrode 606, and counter electrode 610 are placed in contact with solution 612, for example a phosphate buffer solution (pH=6.5) containing hydrogen peroxide, in a container 614 that also includes hydrogen peroxide. Accordingly, working electrode 602 produces a current, which is read by the current meter 604. The amount of current reflects the hydrogen peroxide concentration in the solution 612.

The current meter 604 and operational amplifier 608 can be part of an integrated circuit used to read the sensed hydrogen peroxide concentration. The integrated circuit can be coupled to an output to display the sensed hydrogen peroxide concentration. In order to determine the amount of current corresponding to a particular hydrogen peroxide concentration, after the sensor is produced, the sensor can be calibrated using a number of different hydrogen peroxide concentrations and the corresponding current measurements can be recorded.

A wearable glucose sensor including working electrodes comprising an MXene/Prussian blue composite and an enzyme will now be described in connection with FIGS. 7A-7C. Referring first to FIG. 7A, which illustrates a wearable glucose sensor without a protective cover, the sensor includes a reference electrode 702, counter electrode 704, and a plurality of working electrodes 706, all of which arranged on a substrate 708. In an embodiment, the substrate can be, for example, a silicon substrate. The working electrodes 706 include an MXene/Prussian blue composite, enzyme (e.g., glucose oxidase), and carbon nanotubes arranged on, for example, a carbon fiber membrane, such as carbon fiber paper. In certain instances, the enzyme can cause stress to the film comprising the MXene/Prussian blue composite and carbon nanotubes, which can cause stress cracks in the film. This can be addressed, for example, by laser cutting large pores (˜200 μm) on the surface of the film to release the stress.

Each working electrode can be designed to sense different properties. For example, one working electrode 706 can be provided without an enzyme so that it operates as a pH sensor, one working electrode 706 can be provided with glucose oxidase as an enzyme so that it operates as a glucose sensor, and another working electrode can be provided with lactate oxidase as an enzyme so that it operates as a lactate sensor. This provides for the ability to simultaneously and independently measure different properties using a single sensor. It will be recognized that other enzymes, such as those discussed above, can be employed as an alternative to or in addition to glucose oxidase and lactate oxidase, depending upon is intended to be sensed by the sensor.

The MXene/Prussian blue composite, enzyme, and carbon nanotubes can be arranged on the carbon fiber paper by dissolving a film comprising MXene/Prussian blue composite, enzyme, and carbon nanotubes in, for example, acetone and then transferring the residue to the carbon fiber paper. The reference electrode 702 can comprise, for example, carbon fiber paper and silver/silver chloride, and the counter electrode 704 can comprise, for example, carbon fiber paper and platinum. In an embodiment, the electrodes 702-706 can have a diameter of, for example, 6 mm. The electrodes 702-706 are coupled to a corresponding contact 710 (only one of which is labeled in the figure) via a corresponding lead 712 (only one of which is labeled in the figure). The leads 712 can comprise, for example, liquid metal wires arranged in sealed tunnels. The serpentine tunnels housing the leads 712 can be formed in the substrate 708 by, for example, laser etching. The serpentine shape of the leads 712 is advantageous because it allows the sensor to be stretched and folded. However, the leads 712 can have other shapes, if so desired.

The wearable glucose sensor illustrated in FIG. 7A is a view from the side of the sensor that is intended to contact a person's skin, and accordingly this side of the substrate 708 has an opening 714 so that the sensors 702-706 can contact the person's skin. In contrast, the contacts 710 and leads 712 are not exposed on this side of the substrate 708. The illustration of the opening 714 having a square shape in FIG. 7A is merely an example and the opening can have any shape so long as the electrodes 702-706 can be in contact with a person's skin.

Referring now to FIG. 7B, which illustrates the wearable glucose sensor from the opposite side of the substrate from the view in FIG. 7A, a protective cover 716 is arranged on top of the substrate 708. In an embodiment, the protective cover 716 comprises silicone (for example Ecoflex silicone from Smooth-On, Inc.), which is advantageous because it is stretchable and non-toxic. However, the protective cover 716 can also be comprised of other materials, such as polydimethylsiloxane (PDMS). The protective cover 716 includes external contacts 718 (only one of which is labeled in the figure), which are electrically coupled to the corresponding contacts 710 on the substrate so that the wearable glucose sensor can be electrically coupled to another device for reading the glucose measurements. An opening 720 is formed in the protective cover 716 over each working electrode 706 to allow for contact with air because the enzyme reactions require oxygen as an electron acceptor. The illustration of the openings 720 in FIG. 7B as being circular is merely an example and the openings 720 can have any shape so long as the side of the working electrodes that is opposite to the side contacting a person's skin is in contact with air. The glucose sensor can measure glucose levels by the electrodes 702-706 being contacted by sweat 722 on a person's skin 724. It should be recognized that a portion of the protective cover 716 has been cut-away in FIG. 7B to illustrate the contact of the electrodes with sweat. However, the protective cover 716 will have a contiguous surface except for the openings for the external contacts 718 and the openings 720 above the working electrodes 706.

FIG. 7C is a schematic diagram of a glucose sensor system according to an embodiment. The system includes a glucose sensor 726 electrically coupled to processing electronics 728 via leads 730. The processing electronics 728 includes an integrated circuit providing the voltage source, operational amplifier, and current meter described above in connection with FIG. 6. Moreover, the processing electronics 728 can include a wireless transmitter (or a transceiver if two-way communication is desired) to communicate with an external device 732. In an embodiment, the wireless transmitter (or transceiver) can communicate using, for example, Bluetooth wireless communication technology. Although FIG. 7C illustrates the external device 732 as a smartphone, the external device 732 can be any device that can wirelessly communicate with processing electronics 728. Moreover, in some embodiments, the external device 732 can be physically coupled to the processing electronics 728.

The disclosed glucose sensor can also include a sweat-uptake layer arranged to contact the skin to increase the collection of sweat for sensing. The sweat-uptake layer can include, for example, serpentine tunnels and porous fabric.

The disclosed embodiments provide a hydrogen peroxide sensor, method of forming a hydrogen peroxide sensor, method of using a hydrogen peroxide sensor, and a working electrode for a hydrogen peroxide sensor. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. An electrode, comprising:
a substrate; and
a composite arranged on the substrate, the composite comprising
MXene; and
Prussian blue.

2. The electrode of claim 1, further comprising:
a binder attached to the composite.

3. The electrode of claim 2, wherein the composite comprises a plurality of layers of MXene and Prussian blue and the plurality of layers of MXene and Prussian blue are held together by the binder.

4. The electrode of claim 2, wherein the binder comprises:
carbon nanotubes.

5. The electrode of claim 1, further comprising:
an enzyme arranged on the composite.

6. The electrode of claim 1, wherein the substrate comprises:
carbon fiber.

7. The electrode of claim 1, wherein the MXene is Ti₃C₂Tₓ.

8. The electrode of claim 1, wherein the electrode is configured to detect different concentrations of hydrogen peroxide, H₂O₂, either directly or via a reaction of an enzyme arranged on the composite, the enzyme being glucose oxidase, lactate oxidase, alcohol oxidase, urate oxidase, choline oxidase, or cholesterol oxidase.

9. The electrode of claim 8, wherein the electrode is configured to detect concentrations of hydrogen peroxide greater than or equal to 200 nano Molar.

10. The electrode of claim 1, wherein the composite is a film having a thickness greater than or equal to 0.1 μm and less than or equal to 1 μm.

11. A method of forming an electrode, the method comprising:
forming a composite of MXene and Prussian blue; and
arranging the composite on a substrate.

12. The method of claim 11, further comprising:
mixing MXene, potassium ferricyanide, polyvinylpyrrolidone, and a liquid to form a solution;
heating and then cooling the solution; and
removing precipitate from the cooled solution, wherein the precipitate is the composite of Prussian blue and MXene.

13. The method of claim 12, further comprising:
combining the composite with a binder.

14. The method of claim 13, wherein combining the composite with the binder comprises:
forming a binder solution of nano material and sodium dodecyl sulfate;
mixing the precipitate with the binder solution to form a further solution; and
filtering further precipitate from the further solution to form a film comprising the precipitate.

15. The method of claim 14, further comprising:
transferring the thin film onto the substrate, wherein the substrate is a carbon fiber substrate.

16. A sensor, comprising:
a voltage source (V_{bias});
a reference electrode coupled to the voltage source (V_{bias});
a counter electrode coupled to the voltage source (V_{bias});
a working electrode comprising a substrate and a composite comprising MXene and Prussian blue on the substrate; and
a current meter coupled to the working electrode.

17. The sensor of claim 16, wherein the reference electrode comprises silver, Ag, and silver chloride, AgCl.

18. The sensor of claim 16, further comprising:
a binder attached to the composite.

19. The sensor of claim 18, wherein the composite comprises a plurality of layers of MXene and Prussian blue and the plurality of layers of MXene and Prussian blue are held together by the binder.

20. The sensor of claim 16, further comprising:
an enzyme arranged on the composite.
